(19) United States
(12) Patent Application Publication
(10) Pub. No.: US 2024/0375091 A1
(43) Pub. Date: Nov. 14, 2024

(54) EPOXYDATION CATALYST SYSTEMS AND PROCESS FOR PREPARING EPOXIDES

(71) Applicant: Covestro Deutschland AG, Leverkusen (DE)

(72) Inventors: Matthias Leven, Köln (DE); Jens Langanke, Mechernich (DE)

(21) Appl. No.: 18/569,261

(22) PCT Filed: Jun. 13, 2022
(86) PCT No.: PCT/EP2022/065954

§ 371 Date: Dec. 12, 2023

(30) Foreign Application Priority Data
Jun. 18, 2021 (EP) 21180223.6
Mar. 24, 2022 (EP) 22164095.6

(51) Int. Cl. B01J 27/135; C07D 301/06

(52) U.S. Cl. CPC B01J 27/135; C07D 301/06

(57) ABSTRACT

The invention relates to a first epoxydation catalyst system comprising a mixture of a metal salt of the metals chromium, manganese, molybdenum, lead and/or bismuth and a hydroxide as well as of a redox-active compound. The invention also relates to an additional second epoxydation catalyst system comprising a mixture of an additional metal salt, iodine and a hydroxide. Furthermore, the invention relates to a process for preparing epoxides comprising the oxidative reaction of an alkene in a reactor in the presence of the first epoxydation catalyst system or the second epoxydation catalyst system.

The invention provides a first epoxidation catalyst system comprising a mixture of a metal salt of the metals chromium, manganese, molybdenum, lead and/or bismuth, and a hydroxide, and also a redox-active compound. The invention further provides a further, second epoxidation catalyst system comprising a mixture of a further metal salt, iodine and a hydroxide. Also is a process for producing epoxides, comprising the oxidative conversion of an alkene in a reactor in the presence of the first epoxidation catalyst system or of the second epoxidation catalyst system.

The prior art has described numerous processes for preparing alkylene oxides, specifically propylene oxide, where the chlorohydrin process in particular is of industrial significance, in which propene is reacted with hypochlorous acid or chlorine and water an isomer mixture of 1-chloro-2-propanol and 2-chloro-1-propanol, which is subsequently reacted with milk of lime to give propylene oxide and calcium chloride. However, the formation of a CaCl2) salt burden results in high wastewater contamination or an additional recycling step and incorporation into a chlor-alkali operation. This technology is rated as disadvantageous in terms of carbon footprint and greenhouse gas emissions (Reduction of GHG Emissions in Propylene Oxide Production, Approved VCS Methodology VM0023 Version 1.0, Sep. 9, 2013 Sectoral Scope 5, South Pole Carbon Asset Management Ltd.).

Additionally of relevance is the preparation of propylene oxide by means of coproduct-based methods (e.g. oxirane method), in which ethylbenzene or isobutane is converted in a 1st stage in the presence of oxygen to the respective hydroperoxides, which can then be reacted with propene to give propylene oxide and 1-phenylethanol or tert-butanol as further coproducts. These coproducts can subsequently be converted further to styrene and isobutene or isobutane. However, aside from the propylene oxide, a coproduct is likewise formed, which additionally has to be removed and processed further in further plants. The coproduct-based methods always require a sales market for the coproduct, and make the economics of such processes complex and prone to adverse effects. Thus, as well the technically demanding processes, there are also obvious market-economic risks, and so a technological and economic assessment of these processes does not show a very advantageous overall picture. This is especially because the demand/sales situation for both products, PO and the coproduct, are generally different regionally. In other words, long-range logistics are needed, which entail direct costs and further disadvantages, such as CO2/GHG emissions. The industrial MTBE process requires incorporation into a refinery operation in order to make a maximum economic contribution.

Also of relevance on an industrial scale is what is called the HPPO process, in which propylene is reacted with hydrogen peroxide to give propylene oxide and water. What is advantageous over the aforementioned industrial scale preparation processes is that no coproduct or salt burden arises in the product. However, hydrogen peroxide has to be produced in an upstream, technically complex catalytic process. The process is considered to be technically demanding and is generally operated only at integrated sites with other H2O2 consumers.

The direct oxidation of propene to propylene oxide is additionally considered to be technically immature, and, according to the present state of the art, there is no direct oxidation process which is performable economically on an industrial scale. Particularly the reaction regime and especially the control of temperature in gas phase and salt melt processes are an unsolved challenge, even though a high propene conversion in conjunction with a high PO selectivity is crucial for an efficient industrial process. In the case of direct oxidation, a number of by-products and conversion products, for example methanol, acetaldehyde, carbon dioxide, ethylene and formaldehyde, occur in substantial amounts (cf. D. Kahlich et al., Dow Deutschland in Ullmann's Encyclopedia of Industrial Chemistry, chapter entitled “Propylene Oxide”, Wiley VCH, 2012). For example, current prior art documents that may be cited in respect of the aerobic oxidation of alkenes such as propene are US2018208569A1 and US2020346193A1. The respective epoxy selectivities are only 54.5% and 60%, with respective conversions of 8.6% and 5%.

It was therefore an object of the present invention to provide a catalyst system for the direct production of epoxides, preferably of propylene oxide, which, in the oxidative conversion of alkenes, preferably with oxygen or an oxygen-containing gas mixture, has a with improved epoxide conversion compared to systems known from the prior art and improved product selectivity via reduction in the formation of unwanted by-products and the avoidance of formation of any coproducts. This improved catalyst activity and selectivity should be reflected by a minimum activation energy EA for the reaction sequence or reaction mechanism of the hydroxylation of propene, i.e. the addition of propene onto the catalyst system, and the reductive elimination of the propylene oxide formed from the catalyst system.

It has been found that, surprisingly, the problem addressed by the invention is solved by an epoxidation catalyst system (1), an epoxidation catalyst system (2), and processes for producing epoxides comprising the oxidative conversion of an alkene in a reactor in the presence of the epoxidation catalyst system (1) or the epoxidation catalyst system (2).

The epoxidation catalyst system (1) here comprises
a) a mixture (1) or a reaction product (1) of
   a-1) a metal salt (A) of the metals chromium (Cr), manganese (Mn), molybdenum (Mo), lead (Pb) and/or bismuth (Bi) and
   a-2) a hydroxide (B)
b) a redox-active compound (C).

According to the invention, one or more metal salts (A) of the metals chromium (Cr), manganese (Mn), molybdenum (Mo), lead (Pb) and/or bismuth (Bi) are used.

The embodiments that follow may be combined as desired, unless the opposite is apparent from the technical context and common art knowledge.

In one embodiment of the invention, the metal salt (A) is a nitrate, halide, tetrafluoroborate, sulfate, paratoluenesulfonate, methanesulfonate and/or triflate, preferably a chloride.

In a preferred embodiment of the invention, the metal salt (A) is one or more compounds and is selected from the group consisting of MoCl₅, MoOCl₃, MnCl₂, K₂MnCl₆, CrOCl₂, PbCl₄ and BiCl₃.

In one embodiment of the invention, the hydroxide (B) is an organic hydroxide (B-1) and/or inorganic hydroxide (B-2).

In a preferred embodiment of the invention, the hydroxide (B) is an organic hydroxide (B-1), and the organic hydroxide (B-1) is one or more compound(s) and is selected from the group consisting of 3-(trifluoromethyl)phenyltrimethylammonium hydroxide, tetra-n-butylphosphonium hydroxide, choline hydroxide, benzyltrimethylammonium hydroxide, tetraethylammonium hydroxide, tetra-n-propylammonium hydroxide, tetrabutylammonium hydroxide and tetramethylammonium hydroxide.

In a less preferred, alternative embodiment of the invention, the hydroxide (B) is an inorganic hydroxide (B-2), and the inorganic hydroxide (B-2) is one or more compound(s) and is selected from the group consisting of lithium hydroxide, sodium hydroxide, potassium hydroxide and the crown ether complexes thereof, cesium hydroxide, calcium hydroxide, barium hydroxide, potassium aluminates, sodium aluminate, potassium aluminate and sodium zincate, preferably lithium hydroxide, sodium hydroxide and potassium hydroxide.

In one embodiment of the invention, the redox-active compound (C) is one or more compound(s) and is selected from the group consisting of CuCl₂, Cu(BF₄)₂, CuCl, VCl₃, VOCl₃, NH₄VO₃, 1,4-benzoquinone, 1,4-naphthoquinone, Se₂O₅, TeO₂, TeO₂, Sb₂O₃, Sb₂O₅, CeCl₃, Co(salen), Co(OAc)₂, SnSO₄, Fe(acac)₃, Mo(acac)₃, K₂Cr₂O₇, Mn(OAc)₃, Ni(CF₃CO₂H)₂ and BiCl₃.

In one embodiment of the invention, the calculated molar ratio of the metal salt (A) to the hydroxide groups of the hydroxide (B) for the epoxidation catalyst system (1) is from 1.000 mol:1.000 mol to 6.000 mol:1.000 mol.

In one embodiment of the invention, the calculated molar ratio of the metal salt (A) to the redox-active compound (C) for the epoxidation catalyst system (1) is from 0.100 mol:1.000 mol to 10.000 mol:1.000 mol.

In one embodiment of the invention, the calculated molar ratio of the hydroxide groups of the hydroxide (B) to the redox-active compound (C) for the epoxidation catalyst system (1) is from 0.100 mol:1.000 mol to 50.000 mol:1.000 mol.

In a less preferred embodiment of the invention, the epoxidation catalyst system (1) comprises the mixture (1) of the metal salt (A) and the hydroxide (B) in the above-described calculated molar ratios.

In a preferred embodiment of the invention, the epoxidation catalyst system (1) comprises the reaction product (1) of the metal salt (A) and the hydroxide (B), where the metal salt (A) and the hydroxide (B) are produced by a production method known to the person skilled in the art, for example solid-state syntheses, precipitation or coprecipitation methods, or as in situ salt metathesis. For example, the reaction product (1) can be synthesized by adding the organic hydroxide (B-1) and/or inorganic hydroxide (B-2) to a solution of the nitrate, halide, tetrafluoroborate, sulfate, paratoluenesulfonate, methanesulfonate and/or triflate, preferably a chloride, of the metal salt (A) in the above-described calculated molar ratios, followed by removal of the precipitation product, drying and suitable thermal treatment.

In a preferred embodiment of the invention, the reaction product (1) of the epoxidation catalyst system (1) has a structure of formula (I), (II) and/or (III):

| | | |
|---|---|---|
| (Q R₁R₂R₃R₄)⁺ₙ₊ₒ₋ₘ[M(A)^{m+}(Hal)ₙ(OH)ₒ(S)ₚ]^{m-n-o} | (I) | if m < n + o |
| [M(A)^{m+}(Hal)ₙ(OH)ₒ(S)ₚ] | (II) | if m = n + o |
| [M(A)^{m+}(Hal)ₙ(OH)ₒ(S)ₚ]^{(m-n-o)} [X]⁻ₙ₊ₒ₋ₘ | (III) | if m > n + o |

with
Q=nitrogen or phosphorus, preferably nitrogen,
R₁, R₂, R₃, R₄ independently selected from the group consisting of
   (i) linear or branched alkyl groups containing 1 to 22 carbon atoms, optionally substituted by heteroatoms and/or heteroatom-containing substituents;
   (ii) cycloaliphatic groups containing 3 to 22 carbon atoms with 1 to 3 bridging carbon atoms, optionally substituted by heteroatoms and/or heteroatom-containing substituents, and/or
   (iii) aryl groups containing 6 to 18 carbon atoms, optionally substituted by 1 to 10 carbon atoms and/or optionally substituted by heteroatoms and/or heteroatom-containing substituents;
preferably R₁, R₂, R₃ and R₄ independently selected from the group consisting of methyl, ethyl, isopropyl, n-propyl, isobutyl, t-Bu, n-butyl, phenyl, benzyl, (trifluoromethyl)phenyl,
M(A)^{m+}=Mo⁵⁺, Mn⁴⁺, Cr⁴⁺, Pb⁴⁺, Bi⁵⁺, preferably Mo⁵⁺;
Hal=Cl⁻, Br⁻ or I⁻, preferably Cl⁻ or Br⁻, more preferably Cl⁻;
S=H₂O, THF (tetrahydrofuran), or dioxane, bis(2-methoxyethyl) ether (diglyme), methoxyethanol, polyethylene glycols, pyridine, lutidine, 2,2′-bipyridine, acetonitrile, dimethyl sulfoxide, sulfolane, thiophene, preferably THF;
n+o+p=6
n≥1; preferably n≥2; more preferably n=2
o≥1; preferably o≥1; more preferably o=1; 2; 3 or 4
X=OTf, BF₄⁻, Hal⁻

In a particularly preferred embodiment of the invention, the reaction product (1) of the epoxidation catalyst system (1) is one or more compounds and is selected from the list consisting of [Mo(V)Cl₂(OH)₂(THF)₂]Cl, [Mn(IV)Cl(OH)₃(THF)₂], [Cr(VI)Cl₂(OH)₂(THF)₂]Cl₂, [Pb(IV)Cl₂(OH)₂(THF)₂] and [Bi(V)Cl₂(OH)₃THF].

The blending of the mixture (1) or the reaction product (1) with the redox active compound (C) to give the epoxidation catalyst system (1) is possible by the mixing methods known to the person skilled in the art, for example stirring-in of solutions or suitable blending of solids.

In one embodiment of the invention, the epoxidation catalyst system (1) also contains iodine (I₂).

In a preferred embodiment of the invention, the iodine (I₂) is used in a calculated molar proportion of 1 mol % to 2000 mol % based on the amount of the metal salt (A).

In one embodiment of the invention, the epoxidation catalyst system (1) is applied to a catalyst support (D) to form a supported epoxidation catalyst system (1).

In a preferred embodiment of the invention, the catalyst support (D) is one or more compound(s) and is selected from the group consisting of metal oxides, alkaline earth metal carbonates, silicates, silicon carbides, silicon oxycarbides, silicon nitrides, silicon oxynitrides and silicon dioxide, preferably aluminum oxide aluminum dioxide, silica, titanium dioxide, zirconium dioxide, calcium carbonate, phyllosilicates, such as talc, kaolinites and pyrophyllites, and titanium dioxide.

According to the invention, the catalyst support may take the form of shaped bodies or of powder.

In one embodiment of the invention, the epoxidation catalyst system (1) may be applied to the catalyst support (D) in a calculated proportion by mass of 1.0% by weight to 30.0% by weight.

In one embodiment of the invention, the epoxidation catalyst system (1) is applied to the catalyst support (D) by the wet infiltration method or the incipient wetness method.

The invention further provides an epoxidation catalyst system (2) comprising
c) a mixture (2) or a reaction product (2) of
   c-1) a metal salt (E),
   c-2) iodine (I₂) and
   c-3) a hydroxide (F)
d) optionally a redox-active compound (G).

In one embodiment of the invention, the metal salt (E) is a nitrate, halide, tetrafluoroborate, sulfate, paratoluenesulfonate, methanesulfonate and/or triflate, preferably a chloride.

In a preferred embodiment of the invention, the metal salt (E) is one or more compound(s) and is selected from the group consisting of NiCl₂, MnCl₂, PbCl₂, SnCl₂, CrCl₃, VCl₃, MoCl₄, FeCl₂ and RuCl₃.

In one embodiment of the invention, the hydroxide (F) is an organic hydroxide (F-1) and/or inorganic hydroxide (F-2).

In a preferred embodiment of the invention, the hydroxide (F) is an organic hydroxide (B-1), and the organic hydroxide (F-1) is one or more compound(s) and is selected from the group consisting of 3-(trifluoromethyl)phenyltrimethylammonium hydroxide, tetra-n-butylphosphonium hydroxide, choline hydroxide, benzyltrimethylammonium hydroxide, tetraethylammonium hydroxide, tetra-n-propylammonium hydroxide, tetrabutylammonium hydroxide and tetramethylammonium hydroxide.

In a less preferred, alternative embodiment of the invention, the hydroxide (F) is an inorganic hydroxide (F-2), and the inorganic hydroxide (F-2) is one or more compound(s) and is selected from the group consisting of lithium hydroxide, sodium hydroxide, potassium hydroxide and the crown ether complexes thereof, cesium hydroxide, calcium hydroxide, barium hydroxide, potassium aluminates, sodium aluminate, potassium aluminate and sodium zincate, preferably lithium hydroxide, sodium hydroxide and potassium hydroxide.

In one embodiment of the invention, the redox-active compound (G) is one or more compound(s) and is selected from the group consisting of CuCl₂, Cu(BF₄)₂, CuCl, VCl₃, VOCl₃, NH₄VO₃, 1,4-benzoquinone, 1,4-naphthoquinone, Se₂O₅, TeO₂, TeO₂, Sb₂O₃, Sb₂O₅, CeCl₃, Co(salen), Co(OAc)₂, SnSO₄, Fe(acac)₃, Mo(acac)₃, K₂Cr₂O₇, Mn(OAc)₃, Ni(CF₃CO₂H)₂ and BiCl₃.

In one embodiment of the invention, the calculated molar ratio of the metal salt (E) to the hydroxide groups of the hydroxide (F) for the epoxidation catalyst system (2) is from 1.000 mol:1.000 mol to 6.000 mol:1.000 mol.

In one embodiment of the invention, the calculated molar ratio of the metal salt (E) to the redox-active compound (G) for the epoxidation catalyst system (2) is from 0.100 mol:1.000 mol to 10.000 mol:1.000 mol.

In one embodiment of the invention, the calculated molar ratio of the hydroxide groups of the hydroxide (F) to the redox-active compound (G) for the epoxidation catalyst system (2) is from 0.100 mol:1.000 mol to 50.000 mol:1.000 mol.

In a preferred embodiment of the invention, the iodine (I₂) is used in a calculated molar proportion of 1 mol % to 2000 mol % based on the amount of the metal salt (E).

In a less preferred embodiment of the invention, the epoxidation catalyst system (2) comprises the mixture (2) of the metal salt (E), iodine and the hydroxide (F) in the above-described calculated molar ratios.

In a preferred embodiment of the invention, the oxidation catalyst system (2) comprises the reaction product (2) of the metal salt (E), iodine and the hydroxide (F), where the metal salt (E), the iodine and the hydroxide (F) are produced by a production method known to the person skilled in the art, for example solid-state syntheses, precipitation or coprecipitation methods, or as in situ salt metathesis. For example, the reaction product can be synthesized by adding the organic hydroxide (F-1) and/or inorganic hydroxide (F-2) to a solution of the nitrate, halide, tetrafluoroborate, sulfate, paratoluenesulfonate, methanesulfonate and/or triflate, preferably a chloride, of the metal salt (E) in the above-described calculated molar ratios, followed by removal of the precipitation product, drying and suitable thermal treatment.

In a preferred embodiment of the invention, the reaction product (2) of the epoxidation catalyst system (2) has a structure of formula (IV), (V) and/or (VI):

| | | |
|---|---|---|
| (Q R₁R₂R₃R₄)⁺_{Im-o-pI} [M(E)^{m+}(Hal)ₙ(OH)ₒ(S)ₚ]^{m-n-o} · q · I₂ | (IV) | if m < n + o, |
| [M(E)^{m+}(Hal)ₙ(OH)ₒ(S)ₚ] · q · I₂ | (V) | if m = n + o |
| [M(E)^{m+}(Hal)ₙ(OH)ₒ(S)ₚ]^{m-n-o} · q · I₂ [X]⁻ₙ₊ₒ₋ₘ | (VI) | if m > n + o |

with
Q=nitrogen or phosphorus, preferably nitrogen,
R₁, R₂, R₃, R₄ independently selected from the group consisting of
   (i) linear or branched alkyl groups containing 1 to 22 carbon atoms, optionally substituted by heteroatoms and/or heteroatom-containing substituents;
   (ii) cycloaliphatic groups containing 3 to 22 carbon atoms with 1 to 3 bridging carbon atoms, optionally substituted by heteroatoms and/or heteroatom-containing substituents, and/or
   (iii)aryl groups containing 6 to 18 carbon atoms, optionally substituted by 1 to 10 carbon atoms and/or optionally substituted by heteroatoms and/or heteroatom-containing substituents; preferably R₁, R₂, R₃ and R₄ independently selected from the group consisting of methyl, ethyl, isopropyl, n-propyl, isobutyl, t-Bu, n-butyl, phenyl, benzyl, (trifluoromethyl)phenyl, more preferably methyl, benzyl and n-butyl;
      M(E)^{m+}=Ni²⁺, Mn²⁺, Pb²⁺, Sn²⁺, Cr³⁺, V³⁺, Mo⁴⁺, Fe²⁺ or Ru³⁺, preferably Ni²⁺, Mn²⁺, Pb²⁺, Sn²⁺;
      Hal=Cl⁻, Br⁻ or I⁻, preferably Cl⁻ or Br⁻, more preferably Cl⁻,
      S=H₂O, THF (tetrahydrofuran), or dioxane, bis(2-methoxyethyl) ether (diglyme), methoxyethanol, polyethylene glycols, pyridine, lutidine, 2,2′-bipyridine, acetonitrile, dimethyl sulfoxide, sulfolane, thiophene, preferably THF;
      n+o+p=6;
      n≥1; preferably n≥2; more preferably n=2;
      o≥1; preferably o≥1; more preferably o=1; 2; 3 or 4;
      X=OTf, BF₄⁻, Hal⁻;
      q≥1, preferably 1≤q≤2000, more preferably 1≤q≤10.

In a particularly preferred embodiment of the invention, the reaction product (2) of the epoxidation catalyst system (2) is one or more compounds and is selected from the list consisting of (Q R₁R₂R₃R₄)[Ni(II)Cl₂(OH)(THF)₃] I₂, (Q R₁R₂R₃R₄)[Mn(II)Cl₂(OH)(THF)₃] I₂, (Q R₁R₂R₃R₄)[Pb(II)Cl₂(OH)(THF)₃] I₂ and (Q R₁R₂R₃R₄)[Sn(II)Cl₂(OH)(THF)₃] I₂ with Q=nitrogen and R₁, R₂, R₃ and R₄ independently selected from the group consisting methyl, benzyl and n-butyl. In one embodiment of the invention, the epoxidation catalyst system (2) is applied to a catalyst support (H) to form a supported epoxidation catalyst system (2).

In a preferred embodiment of the invention, the catalyst support (H) is one or more compound(s) and is selected from the group consisting of metal oxides, alkaline earth metal carbonates, silicates, silicon carbides, silicon oxycarbides, silicon nitrides, silicon oxynitrides and silicon dioxide, preferably aluminum oxide aluminum dioxide, silica, titanium dioxide, zirconium dioxide, calcium carbonate, phyllosilicates, such as talc, kaolinites and pyrophyllites, and titanium dioxide.

According to the invention, the catalyst support may take the form of shaped bodies or of powder.

In one embodiment of the invention, the epoxidation catalyst system (2) is applied to the catalyst support (H) in a calculated proportion by mass of 1.0% by weight to 30.0% by weight.

In one embodiment of the invention, the epoxidation catalyst system (H) is applied to the catalyst support (H) by the wet infiltration method or the incipient wetness method.

The present invention further provides a process for producing epoxides, comprising the oxidative conversion of an alkene in a reactor in the presence of the epoxidation catalyst system (1) of the invention or of the epoxidation catalyst system of the invention.

In one embodiment of the process of the invention, the oxidative conversion in the reactor is effected in the presence of oxygen or an oxygen-containing gas mixture.

The oxygen-containing gas mixture, as well as oxygen, also comprises diluent, carrier or inert gases such as hydrocarbons, noble gases, CO, CO2, N2.

In one embodiment of the process of the invention, further additives are added to the oxygen or an oxygen-containing gas mixture, such as water, O, N-containing compounds, for example hydrazine, ammonia (NH3), methylamine (MeNH₂), NOx, PH₃, SO₂ and/or SO₃, in amounts of 10 ppm to 500 ppm, preferably of 30 ppm to 300 ppm and more preferably of 50 ppm to 200 ppm. In addition, CO2 may be added in a proportion of 0.01% by volume to 50% by volume, preferably of 0.1% by volume to 20% by volume, and more preferably of 1% by volume to 10% by volume. It is also possible to add organic halides, such as ethylene dichloride, ethyl chloride, vinyl chloride, methyl chloride and/or methylene chloride, in amounts of 10 ppm to 500 ppm, preferably of 50 ppm to 400 ppm, and more preferably of 100 ppm to 300 ppm.

In a preferred embodiment of the process of the invention, the oxidative conversion in the reactor is effected in the presence of oxygen.

In one embodiment of the process of the invention, the alkene is one or more compound(s) and is selected from the group consisting of ethene, propene, butene, 1-octene, butadiene, butane-1,4-diol diallyl ether, allyl chloride, allyl alcohol, styrene, cyclopentene, cyclohexene, phenyl allyl ether, diallyl ether, n-butyl allyl ether, tert-butyl allyl ether, bisphenol A diallyl ether, resorcinol diallyl ether, triphenylolmethane triallyl ether, cyclohexane-1,2-dicarboxylic acid bis(allyl ester), isocyanuric acid tris(prop-2,3-ene) ester and mixtures of these alkenes, preferably ethene, propene and allyl chloride, and more preferably propene.

The epoxide (alkylene oxide) of the invention may be an epoxide having 2-45 carbon atoms. In one embodiment of the process of the invention, the epoxide is selected from at least one compound from the group consisting of ethylene oxide, propylene oxide, 1,2-butylene oxide, 2,3-butylene oxide, 2-methyl-1,2-propylene oxide (isobutene oxide), 1,2-pentylene oxide, 2,3-pentylene oxide, 2-methyl-1,2-butylene oxide, 3-methyl-1,2-butylene oxide, epoxides of C6-C22 α-olefins, such as 1,2-hexylene oxide, 2,3-hexylene oxide, 3,4-hexylene oxide, 2-methyl-1,2-pentylene oxide, 4-methyl-1,2-pentylene oxide, 2-ethyl-1,2-butylene oxide, 1,2-heptylene oxide, 1,2-octylene oxide, 1,2-nonylene oxide, 1,2-decylene oxide, 1,2-undecylene oxide, 1,2-dodecylene oxide, 4-methyl-1,2-pentylene oxide, cyclopentylene oxide, cyclohexylene oxide, cycloheptylene oxide, cyclooctylene oxide, styrene oxide, methylstyrene oxide, pinene oxide, allyl glycidyl ether, vinylcyclohexylene oxide, cyclooctadiene monoepoxide, cyclododecatriene monoepoxide, butadiene monoepoxide, isoprene monoepoxide, limonene oxide, 1,4-divinylbenzene monoepoxide, 1,3-divinylbenzene monoepoxide, glycidyl acrylate and glycidyl methacrylate, mono- or polyepoxidized fats as mono-, di- and triglycerides, epoxidized fatty acids, C1-C24 esters of epoxidized fatty acids, epichlorohydrin, glycidol, and derivatives of glycidol, for example glycidyl ethers of C1-C22 alkanols and glycidyl esters of C1-C22 alkanecarboxylic acids. Examples of derivatives of glycidol are phenyl glycidyl ether, cresyl glycidyl ether, methyl glycidyl ether, ethyl glycidyl ether and 2-ethylhexyl glycidyl ether.

In a preferred embodiment of the process, the epoxide is ethylene oxide, propylene oxide, 1,2-butylene oxide, 1,2-pentylene oxide, 1,2-hexylene oxide, 1,2-heptylene oxide and/or 1,2-octylene oxide. In a particularly preferred embodiment of the process, the epoxide is ethylene oxide and/or propylene oxide. In a very particularly preferred embodiment of the process, the epoxide is propylene oxide.

In one embodiment of the process of the invention, epoxides are produced in the presence of a solvent.

In one embodiment of the process of the invention, the solvent is one or more compound(s) and is selected from the group consisting of CO2, water, perfluoromethyldecalin, perfluorodecalin, perfluoroperhydrophenanthrene, perfluoro(butyltetrahydrofuran), tetrahydrofuran, 2-methyl-THF, acetic acid, acetonitrile, dimethyl sulfoxide, sulfolane, acetone, ethyl methyl ketone, dimethylformamide, dichloromethane, chloroform, tetrachloromethane, N-methyl-2-pyrrolidinone, methyl t-butyl ether (MTBE), dimethyl sulfoxide (DMSO), hexamethylphosphoramide, dichlorobenzene, 1,2-dichloroethylene, 1,1,1,3,3,3-hexafluoroisopropanol, perfluoro-tert-butyl alcohol, 1,1,2,3,3-pentafluoropropane, 1-bromo-2-chloro-1,1,2-trifluoroethane, 1,2-dichloro-1,1,2,3,3,3-hexafluoropropane, ethylene glycol, glycerol, and phenol.

In one embodiment of the process of the invention, epoxides are produced in the presence of a solvent, where the production at a temperature of 20° C. to 200° C., preferably of 50° C. to 160° C. and more preferably of 100° C. to 150° C.

In one embodiment of the process of the invention, epoxides are produced in the presence of a solvent, where the production is effected at a pressure of 1 bara to 200 bara, preferably of 1 bara to 35 bara and more preferably of 1 bara to 28 bara.

In one embodiment of the process of the invention, epoxides are produced in the presence of a solvent, where the production is effected within a period of 6 min to 48 h, preferably of 6 min to 24 h and more preferably of 6 min to 3 h.

In one embodiment of the process of the invention, epoxides are produced in the presence of a solvent, where the molar ratio of the alkene to oxygen is from 1:100 to 100:1, preferably from 1:30 to 30:1.

In an alternative embodiment of the process of the invention, epoxides are produced in the absence of a solvent.

What is meant here in accordance with the invention by a solvent-free process is that solvent residues, for example as a result of the production of the feedstocks, may be present at up to 10% by volume, preferably up to 5% by volume and more preferably up to 2% by volume, based on the amount of propene used.

In one embodiment of the process of the invention, epoxides are produced in the absence of a solvent, where the production is effected using the supported epoxidation catalyst system (1) of the invention or the supported epoxidation catalyst system (2) of the invention.

In one embodiment of the process of the invention, epoxides are produced in the absence of a solvent, where the production is effected at a temperature of 20° C. to 500° C., preferably of 50° C. to 400° C. and more preferably of 50° C. to 250° C.

In one embodiment of the process of the invention, epoxides are produced in the absence of a solvent, where the production is effected at a pressure of 1 bara to 200 bara, preferably of 2 bara to 100 bara and more preferably of 2 bara to 50 bara.

In one embodiment of the process of the invention, epoxides are produced in the absence of a solvent, where the production is effected at a gas hourly space velocity (GHSV) as the quotient of the volume flow rate of the reactant gases used to the catalytic volume of 100 h-1 to 10 000 h-1, preferably of 200 h-1 to 5000 h-1 and more preferably of 500 h-1 to 2000 h-1. The catalyst volume corresponds here, in the case of supported catalysts and/or catalysts diluted by means of inert diluent (for example aluminum oxide, silica, silicon carbide), to that of the supported epoxidation catalyst system (1) or (2) and/or to the sum total of the catalyst volume and diluent volume.

In one embodiment of the process of the invention, epoxides are produced in the absence of a solvent, where the molar ratio of the alkene to oxygen is from 1.0:0.1 to 2:1, preferably from 1:0.5 to 2:1 and more preferably from 1:0.8 to 2:1.

In one embodiment of the process of the invention, the alkene is metered into the reactor continuously or stepwise, preferably continuously.

In one embodiment of the process of the invention, oxygen or the oxygen-containing gas mixture is metered into the reactor continuously or stepwise, preferably continuously.

In one embodiment of the process of the invention, the alkene and the oxygen or the oxygen-containing gas mixture is metered into the reactor continuously or stepwise, preferably continuously.

In a preferred embodiment of the process of the invention, the epoxide is withdrawn from the reactor continuously or stepwise, preferably continuously.

In a preferred embodiment of the process of the invention, the epoxidation catalyst system (1) is metered into the reactor continuously or stepwise, preferably continuously.

The process of the invention may be performed in a batchwise process, in a semi-batchwise process or in continuous mode, for which reactor types known to the person skilled in the art are used. Thus, the continuous production of epoxides can be effected by oxidative conversion of alkenes in a continuously backmixed stirred tank (continuous stirred tank reactor, CSTR), or in a one- or two-column bubble reactor in the liquid phase in the presence of a solvent. The continuous production of epoxides in the gas phase in the absence of a solvent can be effected by means of a continuous gas phase reactor, for example a fixed bed reactor, also with use of ceramic filler materials.

In a preferred embodiment of the process of the invention, the epoxidation catalyst system (1) and/or epoxidation catalyst system (2) is used in a calculated amount of 10 ppm to 500 000 ppm, preferably of 100 ppm to 200 000 ppm and more preferably from 1000 ppm to 150 000 ppm, based on the amount of all reactants involved and optionally solvents and possible further auxiliaries.

In a first embodiment, the invention relates to an epoxidation catalyst system (1) comprising
a) a mixture, preferably a reaction product, of
   a-1) a metal salt (A) of the metals chromium (Cr), manganese (Mn), molybdenum (Mo), lead (Pb) and/or bismuth (Bi) and
   a-2) a hydroxide (B)
b) a redox-active compound (C).

In a second embodiment, the invention relates to an epoxidation catalyst system (1) according to the first embodiment, wherein the metal salt (A) is a nitrate, halide, tetrafluoroborate, sulfate, paratoluenesulfonate, methanesulfonate and/or triflate, preferably a chloride.

In a third embodiment, the invention relates to an epoxidation catalyst system (1) according to the first or second embodiment, wherein the metal salt (A) is one or more compounds and is selected from the group consisting of MoCl₅, MoOCl₃, MnCl₂, K₂MnCl₆, CrOCl₂, PbCl₄ and BiCl₃.

In a fourth embodiment, the invention relates to an epoxidation catalyst system (1) according to the first to third embodiments, wherein the hydroxide (B) is an organic hydroxide (B-1) and/or inorganic hydroxide (B-2).

In a fifth embodiment, the invention relates to an epoxidation catalyst system (1) according to the fourth embodiment, wherein the hydroxide (B) is an organic hydroxide (B-1), and the organic hydroxide (B-1) is one or more compound(s) and is selected from the group consisting of 3-(trifluoromethyl)phenyltrimethylammonium hydroxide, tetra-n-butylphosphonium hydroxide, choline hydroxide, benzyltrimethylammonium hydroxide, tetraethylammonium hydroxide, tetra-n-propylammonium hydroxide, tetrabutylammonium hydroxide and tetramethylammonium hydroxide.

In a sixth embodiment, the invention relates to an epoxidation catalyst system (1) according to the fourth embodiment, wherein the hydroxide (B) is an inorganic hydroxide (B-2), and the inorganic hydroxide (B-2) is one or more compound(s) and is selected from the group consisting of lithium hydroxide, sodium hydroxide, potassium hydroxide and the crown ether complexes thereof, cesium hydroxide, calcium hydroxide, barium hydroxide, potassium aluminates, sodium aluminate, potassium aluminate and sodium zincate, preferably lithium hydroxide, sodium hydroxide and potassium hydroxide.

In a seventh embodiment, the invention relates to an epoxidation catalyst system (1) according to the first to sixth embodiments, wherein the redox-active compound (C) is one or more compound(s) and is selected from the group consisting of copper dichloride, CuCl₂, Cu(BF₄)₂, CuCl, VCl₃, VOCl₃, NH₄VO₃, 1,4-benzoquinone, 1,4-naphthoquinone, Se₂O₅, TeO₂, TeO₂, Sb₂O₃, Sb₂O₅, CeCl₃, Co(salen), Co(OAc)₂, SnSO₄, Fe(acac)₃, Mo(acac)₃, K₂Cr₂O₇, Mn(OAc)₃, Ni(CF₃CO₂H)₂ and BiCl₃.

In an eighth embodiment, the invention relates to an epoxidation catalyst system (1) according to one of the first to seventh embodiments, wherein the calculated molar ratio of the metal salt (A) to the hydroxide groups of the hydroxide (B) is from 1.000 mol:1.000 mol to 6.000 mol:1.000 mol.

In a ninth embodiment, the invention relates to an epoxidation catalyst system (1) according to one of the first to eighth embodiments, wherein the calculated molar ratio of the metal salt (A) to the redox-active compound (C) is from 0.100 mol:1.000 mol to 10.000 mol:1.000 mol.

In a tenth embodiment, the invention relates to an epoxidation catalyst system (1) according to one of the first to ninth embodiments, wherein the calculated molar ratio of the hydroxide groups of the hydroxide (B) to the redox-active compound (C) is from 0.100 mol:1.000 mol to 50.000 mol:1.000 mol.

In an eleventh embodiment, the invention relates to an epoxidation catalyst system (1) according to one of the first to tenth embodiments, wherein the epoxidation catalyst (1) also contains iodine (I₂).

In a twelfth embodiment, the invention relates to an epoxidation catalyst system (1) according to the eleventh embodiment, wherein the iodine (I₂) is used in a calculated molar proportion of 1 mol % to 2000 mol % based on the amount of the metal salt (A).

In a thirteenth embodiment, the invention relates to an epoxidation catalyst system (1) according to one of the first to twelfth embodiments, wherein the epoxidation catalyst system (1) is applied to a catalyst support (D) to form a supported epoxidation catalyst system (1).

In a fourteenth embodiment, the invention relates to an epoxidation catalyst system (1) according to the thirteenth embodiment, wherein the catalyst support (D) is one or more compound(s) and is selected from the group consisting of metal oxides, alkaline earth metal carbonates, silicates, silicon carbides, silicon oxycarbides, silicon nitrides, silicon oxynitrides and silicon dioxide, preferably aluminum oxide aluminum dioxide, silica, titanium dioxide, zirconium dioxide, calcium carbonate, phyllosilicates, such as talc, kaolinites and pyrophyllites, and titanium dioxide.

In a fifteenth embodiment, the invention relates to an epoxidation catalyst system (1) according to the thirteenth or fourteenth embodiment, wherein the epoxidation catalyst system (1) is applied to the catalyst support (D) in a calculated proportion by mass of 1.0% by weight to 30.0% by weight.

In a sixteenth embodiment, the invention relates to an epoxidation catalyst system (1) according to one of the thirteenth to fifteenth embodiments, wherein the epoxidation catalyst system (1) is applied to the catalyst support (D) by the wet infiltration method or the incipient wetness method.

In a seventeenth embodiment, the invention relates to an epoxidation catalyst system (2) comprising
c) a mixture, preferably a reaction product, of
   c-1) a metal salt (E),
   c-2) iodine (I₂) and
   c-3) a hydroxide (F)
d) optionally a redox-active compound (G).

In an eighteenth embodiment, the invention relates to an epoxidation catalyst system (2) according to the seventeenth embodiment, wherein the metal salt (E) is a nitrate, halide, tetrafluoroborate, sulfate, paratoluenesulfonate, methanesulfonate and/or triflate, preferably a chloride.

In a nineteenth embodiment, the invention relates to an epoxidation catalyst system (2) according to the seventeenth or eighteenth embodiment, wherein the metal salt (E) is one or more compounds and is selected from the group consisting of NiCl₂, MnCl₂, PbCl₂, SnCl₂, CrCl₃, VCl₃, MoCl₄, FeCl₂ and RuCl₃.

In a twentieth embodiment, the invention relates to an epoxidation catalyst system (2) according to one of the seventeenth to nineteenth embodiments, wherein the hydroxide (F) is an organic hydroxide (F-1) and/or inorganic hydroxide (F-2).

In a twenty-first embodiment, the invention relates to an epoxidation catalyst system (2) according to the twentieth embodiment, wherein the hydroxide (F) is an organic hydroxide (B-1), and the organic hydroxide (F-1) is one or more compound(s) and is selected from the group consisting of 3-(trifluoromethyl)phenyltrimethylammonium hydroxide, tetra-n-butylphosphonium hydroxide, choline hydroxide, benzyltrimethylammonium hydroxide, tetraethylammonium hydroxide, tetra-n-propylammonium hydroxide, tetrabutylammonium hydroxide and tetramethylammonium hydroxide.

In a twenty-second embodiment, the invention relates to an epoxidation catalyst system (2) according to the twenty-first embodiment, wherein the hydroxide (F) is an inorganic hydroxide (F-2), and the inorganic hydroxide (F-2) is one or more compound(s) and is selected from the group consisting of lithium hydroxide, sodium hydroxide, potassium hydroxide and the crown ether complexes thereof, cesium hydroxide, calcium hydroxide, barium hydroxide, potassium aluminates, sodium aluminate, potassium aluminate and sodium zincate, preferably lithium hydroxide, sodium hydroxide and potassium hydroxide.

In a twenty-third embodiment, the invention relates to an epoxidation catalyst system (2) according to one of the seventeenth to twenty-second embodiments, wherein the redox-active compound (G) is present, and the redox-active compound (G) is one or more compound(s) and is selected from the group consisting of CuCl₂, Cu(BF₄)₂, CuCl, VCl₃, VOCl₃, NH₄VO₃, 1,4-benzoquinone, 1,4-naphthoquinone, Se₂O₅, TeO₂, TeO₂, Sb₂O₃, Sb₂O₅, CeCl₃, Co(salen), Co(OAc)₂, SnSO₄, Fe(acac)₃, Mo(acac)₃, K₂Cr₂O₇, Mn(OAc)₃, Ni(CF₃CO₂H)₂ and BiCl₃.

In a twenty-fourth embodiment, the invention relates to an epoxidation catalyst system (2) according to one of the seventeenth to twenty-third embodiments, wherein the calculated molar ratio of the metal salt (E) to the hydroxide groups of the hydroxide (B) is from 1.000 mol:1.000 mol to 6.000 mol:1.000 mol.

In a twenty-fifth embodiment, the invention relates to an epoxidation catalyst system (2) according to one of the seventeenth to twenty-fourth embodiments, wherein the calculated molar ratio of the metal salt (E) to the redox-active compound (G) is from 0.100 mol:1.000 mol to 10.000 mol:1.000 mol.

In a twenty-sixth embodiment, the invention relates to an epoxidation catalyst system (2) according to one of the seventeenth to twenty-fifth embodiments, wherein the calculated molar ratio of the hydroxide groups of the hydroxide (F) to the redox-active compound (G) is from 0.100 mol:1.000 mol to 50.000 mol:1.000 mol.

In a twenty-seventh embodiment, the invention relates to an epoxidation catalyst system (1) according to any of the seventeenth to twenty-sixth embodiments, wherein the iodine (I₂) is used in a calculated molar proportion of 1 mol % to 2000 mol % based on the amount of the metal salt (A).

In a twenty-eighth embodiment, the invention relates to an epoxidation catalyst system (2) according to one of the seventeenth to twenty-seventh embodiments, wherein the epoxidation catalyst system (2) is applied to a catalyst support (H) to form a supported epoxidation catalyst system (2).

In a twenty-ninth embodiment, the invention relates to an epoxidation catalyst system (2) according to the twenty-eighth embodiment, wherein the catalyst support (H) is one or more compound(s) and is selected from the group consisting of metal oxides, alkaline earth metal carbonates, silicates, silicon carbides, silicon oxycarbides, silicon nitrides, silicon oxynitrides and silicon dioxide, preferably aluminum oxide aluminum dioxide, silica, titanium dioxide, zirconium dioxide, calcium carbonate, phyllosilicates, such as talc, kaolinites and pyrophyllites, and titanium dioxide.

In a thirtieth embodiment, the invention relates to an epoxidation catalyst system (2) according to the twenty-eighth or twenty-ninth embodiment, wherein the epoxidation catalyst system (2) is applied to the catalyst support (H) in a calculated proportion by mass of 1.0% by weight to 30.0% by weight.

In a thirty-first embodiment, the invention relates to an epoxidation catalyst system (2) according to one of the twenty-eighth to thirtieth embodiments, wherein the epoxidation catalyst system (2) is applied to the catalyst support (H) by the wet infiltration method or the incipient wetness method.

In a thirty-second embodiment, the invention relates to a process for producing epoxides, comprising the oxidative conversion of an alkene in a reactor in the presence of the epoxidation catalyst system (1) according to any of the first to sixteenth embodiments or of the epoxidation catalyst system (2) according to any of the seventeenth to thirty-first embodiments.

In a thirty-third embodiment, the invention relates to a process according to the thirty-second embodiment, wherein the oxidative conversion in the reactor is effected in the presence of oxygen or an oxygen-containing gas mixture.

In a thirty-fourth embodiment, the invention relates to a process according to the thirty-second or thirty-third embodiment, wherein the alkene is one or more compound(s) and is selected from the group consisting of ethene, propene, butene, 1-octene, butadiene, butane-1,4-diol diallyl ether, allyl chloride, allyl alcohol, styrene, cyclopentene, cyclohexene, phenyl allyl ether, diallyl ether, n-butyl allyl ether, tert-butyl allyl ether, bisphenol A diallyl ether, resorcinol diallyl ether, triphenylolmethane triallyl ether, cyclohexane-1,2-dicarboxylic acid bis(allyl ester), isocyanuric acid tris(prop-2,3-ene) ester and mixtures of these alkenes, preferably ethene, propene and allyl chloride, and more preferably propene.

In a thirty-fifth embodiment, the invention relates to a process according to any of the thirty-second to thirty-fourth embodiments, wherein the production is effected in the presence of a solvent.

In a thirty-sixth embodiment, the invention relates to a process according to the thirty-fifth embodiment, wherein the solvent is one or more compound(s) and is selected from the group consisting of CO2, water, perfluoromethyldecalin, perfluorodecalin, perfluoroperhydrophenanthrene, perfluoro(butyltetrahydrofuran), tetrahydrofuran, 2-methyl-THF, acetic acid, acetonitrile, dimethyl sulfoxide, sulfolane, acetone, ethyl methyl ketone, dimethylformamide, dichloromethane, chloroform, tetrachloromethane, N-methyl-2-pyrrolidinone, methyl t-butyl ether (MTBE), dimethyl sulfoxide (DMSO), hexamethylphosphoramide, dichlorobenzene, 1,2-dichloroethylene, 1,1,1,3,3,3-hexafluoroisopropanol, perfluoro-tert-butyl alcohol, 1,1,2,3,3-pentafluoropropane, 1-bromo-2-chloro-1,1,2-trifluoroethane, 1,2-dichloro-1,1,2,3,3,3-hexafluoropropane, ethylene glycol, glycerol, and phenol.

In a thirty-seventh embodiment, the invention relates to a process according to the thirty-fifth or thirty-sixth embodiment, wherein the production is effected at a temperature of 20° C. to 200° C., preferably of 50° C. to 160° C. and more preferably of 100° C. to 150° C.

In a thirty-eighth embodiment, the invention relates to a process according to one of the thirty-fifth to thirty-seventh embodiments, wherein the production is effected at a pressure of 1 bara to 200 bara, preferably of 1 bara to 35 bara and more preferably of 1 bara to 28 bara.

In a thirty-ninth embodiment, the invention relates to a process according to one of the thirty-fifth to thirty-eighth embodiments, wherein the production is effected within a period of 6 min to 48 h, preferably of 6 min to 24 h, and more preferably of 6 min to 3 h.

In a fortieth embodiment, the invention relates to a process according to any of the thirty-fifth to thirty-ninth embodiments, wherein the molar ratio of the alkene to oxygen is from 1:100 to 100:1, preferably from 1:30 to 30:1.

In a forty-first embodiment, the invention relates to a process according to any of the thirty-second to thirty-fourth embodiments, wherein the production is effected in the absence of a solvent.

In a forty-second embodiment, the invention relates to a process according to the forty-first embodiment, wherein the production using the supported epoxidation catalyst system (1) according to any of the thirteenth to sixteenth embodiments or a supported epoxidation catalyst system (2) according to any of the twenty-eighth to thirty-first embodiments.

In a forty-third embodiment, the invention relates to a process according to the forty-first or forty-second embodiment, wherein the production is effected at a temperature of 20° C. to 500° C., preferably of 50° C. to 400° C. and more preferably of 50° C. to 250° C.

In a forty-fourth embodiment, the invention relates to a process according to one of the forty-first to forty-third embodiments, wherein the production is effected at a pressure of 1 bara to 200 bara, preferably of 2 bara to 100 bara and more preferably of 2 bara to 50 bara.

In a forty-fifth embodiment, the invention relates to a process according to one of the forty-first to forty-fourth embodiments, wherein the production is effected at a space velocity of 100 h-1 to 10 000 h-1, preferably of 200 h-1 to 5000 h-1 and more preferably of 500 h-1 to 2000 h-1.

In a forty-sixth embodiment, the invention relates to a process according to one of the forty-first to forty-fifth embodiments, wherein the molar ratio of the alkene to oxygen is from 1.0:0.1 to 2.0:1.0, preferably from 1.0:0.5 to 2.0:1.0 and more preferably from 1.0:0.8 to 2.0:1.0.

In a forty-seventh embodiment, the invention relates to a process according to any of the thirty-second to forty-sixth embodiments, wherein the alkene is metered into the reactor continuously or stepwise, preferably continuously.

In a forty-eighth embodiment, the invention relates to a process according to any of the thirty-third to forty-seventh embodiments, wherein the oxygen or the oxygen-containing gas mixture is metered into the reactor continuously or stepwise, preferably continuously.

In a forty-ninth embodiment, the invention relates to a process according to any of the thirty-third to forty-eighth embodiments, wherein the alkene and the oxygen or the oxygen-containing gas mixture is metered into the reactor continuously or stepwise, preferably continuously.

In a fiftieth embodiment, the invention relates to a process according to any of the forty-seventh to forty-ninth embodiments, wherein the epoxide is withdrawn from the reactor continuously or stepwise, preferably continuously.

In a fifty-first embodiment, the invention relates to a process according to any of the forty-seventh to fiftieth embodiments, wherein the epoxidation catalyst system (1) and/or epoxidation catalyst system (2) is metered into the reactor continuously or stepwise, preferably continuously.

In a fifty-second embodiment, the invention relates to a process according to any of the thirty-second to fifty-first embodiments, wherein the epoxidation catalyst system (1) and/or epoxidation catalyst system (2) is used in a calculated amount of 10 ppm to 500 000 ppm, preferably of 100 ppm to 200 000 ppm and more preferably from 1000 ppm to 150 000 ppm, based on the amount of all reactants involved and optionally solvents and possible further auxiliaries.

### EXAMPLES

### Starting Materials Used:

Metal chlorides and metal oxides: All metal salts used are sourced directly from commercial sources and used in the corresponding reactions without further processing as precursors for the active catalysts:
molybdenum(V) chloride from the manufacturer Sigma Aldrich Corporation in a purity of 95%;
manganese(II) chloride from the manufacturer Sigma Aldrich Corporation in a purity of 99%;
chromium(VI) oxychloride from the manufacturer Sigma Aldrich Corporation in a purity of 99%;
lead(II) chloride from the manufacturer Sigma Aldrich Corporation in a purity of 98%;
bismuth(III) chloride from the manufacturer Sigma Aldrich Corporation in a purity of 98%;
copper(II) chloride from the manufacturer Sigma Aldrich Corporation in a purity of 97%;
nickel(II) chloride from the manufacturer Sigma Aldrich Corporation in a purity of 98%;
tin(II) chloride from the manufacturer Sigma Aldrich Corporation in a purity of 98%.

Molybdenum(VI) oxychloride was produced in accordance with Vitzthumecker et al. in Monatsh. Chem. 148, 629-633 (2017).

Elemental iodine, Triton B and all solvents used were likewise introduced into the reaction without further processing:
iodine from the manufacturer Sigma Aldrich Corporation in a purity of 99.8%;
tetrahydrofuran (THF) from the manufacturer Sigma Aldrich Corporation in a purity of 99.9% (inhibitor-free);
benzyltrimethylammonium hydroxide (Triton B) from the manufacturer Sigma Aldrich Corporation as a 40% solution in methanol.

Propylene was sourced from the manufacturer Sigma Aldrich Corporation in a purity of 99.9% and used as obtainable.

The chemicals are used for the synthesis without further purification.

### Gas Chromatography Analysis

The gas chromatography analysis (GC for short) of liquid and gas samples was performed in accordance with “Determine Impurities in High-Purity Propylene Oxide with Agilent J&W PoraBOND U” by Dianli Ma, Ningbo ZRCC Lyondell Chemical Co., Ltd Zhejiang, China, and Yun Zou, Hua Wu, Agilent Technologies, Inc. On the basis of GC, conversions of propene, yields of propylene oxides and selectivities were ascertained.

### Simulation Method

All quantum mechanical calculations were carried out using the software package TURBOMOLE version 7.4.1 from Cosmologic GmbH & Co. KG. The density functional theory method used was the TPSS density functional, implemented as unrestricted DFT for spin contamination of open-shell systems, with a def2-SVP basis set, as implemented as standard in the Turbomole software package. The energies obtained were refined by the DFT method described and with a basis set of def2-TZVP quality.

Transition states were calculated by gradient-based Monte Carlo, as described in application WO 2020/079094A2.

### Production of the Epoxidation Catalyst System (1)

The epoxidation catalyst systems listed in tables 1 and 2 were produced and used as described in the general test methods.

### General Procedure for the In Situ Synthesis of the Epoxidation Catalyst System (1)

A reaction vessel with stirrer apparatus and protective gas sparging was initially charged with 200 ml tetrahydrofuran under inert conditions. Added to the solvent in each experiment were 0.020 mol of the metal (oxy)chloride of the appropriate oxidation state (see table 1, 1-5) and 0.020 mol of CuCl₂ as redox-active compound (C) under an opposing protective gas flow. While stirring vigorously, N-benzyltrimethylammonium hydroxide (trade name: Triton B) was in each case stirred in until a liquid sample taken, in contact with a water-moist pH test strip, gave a pH of 10-11.

The suspension thus produced was used without further purification for the oxidation of propene.

TABLE 1
Overview of the epoxidation catalyst systems
(1) used for oxidative conversion of propene

| | Epoxidation catalyst system (1) | | | |
|---|---|---|---|---|
| | Reaction | Metal salt | Hydroxide | Redox-active |
| No. | product (1) | (A) | (B) | compound (C) |
| 1 | [Mo(V)Cl₂(OH)₂(THF)₂] | MoCl₅, | Benzyltrimethyl- | CuCl₂ |
| | Cl | MoOCl₃ | ammonium | |
| | | | hydroxide | |
| 2 | [Mn(IV)Cl(OH)₃(THF)₂] | MnCl₂, | Benzyltrimethyl- | CuCl₂ |
| | | K₂MnCl₆ | ammonium | |
| | | | hydroxide | |
| 3 | [Cr(VI)Cl₂(OH)₂(THF)₂] | CrOCl₂ | Benzyltrimethyl- | CuCl₂ |
| | Cl₂ | | ammonium | |
| | | | hydroxide | |
| 4 | [Pb(IV)Cl₂(OH)₂(THF)₂] | PbCl₄ | Benzyltrimethyl- | CuCl₂ |
| | | | ammonium | |
| | | | hydroxide | |
| 5 | [Bi(V)Cl₂(OH)₃THF] | BiCl₃ | Benzyltrimethyl- | CuCl₂ |
| | | | ammonium | |
| | | | hydroxide | |
| 6 (comp.) | Cu(II)(OH)₂ | CuCl₂ | Benzyltrimethyl- | CuCl₂ |
| | | | ammonium | |
| | | | hydroxide | |
| 7 (comp.) | [Ti(IV)Cl₂(OH)₂(THF)₂] | TiCl₄ | Benzyltrimethyl- | CuCl₂ |
| | | | ammonium | |
| | | | hydroxide | |

(comp.) comparative example

Production of the Epoxidation Catalyst System (2)

### General Procedure for the In Situ Synthesis of the Epoxidation Catalyst System (2)

A reaction vessel with stirrer apparatus and protective gas sparging was initially charged with a mixture of 180 ml tetrahydrofuran and 20 nil of water under inert conditions. Added to the solvent mixture were 0.020 mol of the metal (oxy)chloride of the appropriate oxidation state (see table 2, 9-12) and 0.040 mol (5.1 g) of elemental iodine under an opposing protective gas flow. While stirring vigorously, N-benzyltrimethylammonium hydroxide (trade name: Triton B) was stirred in until a liquid sample taken, in contact with a water-moist pH test strip, gave a pH of 10-11.

The suspension thus produced was used without further purification for the oxidation of propene.

TABLE 2
Overview of the epoxidation catalyst systems
(2) used for oxidative conversion of propene

| | Epoxidation catalyst system (2) | | | |
|---|---|---|---|---|
| | | Metal | Iodine | Hydroxide |
| No. | Reaction product (2) | salt (E) | (I₂) | (F) |
| 8 (comp.) | Q(1)[Pt(II)Cl₂(OH)(THF)₃] | PtCl₂ | I₂ | Benzyltrimethyl- |
| | I₂ | | | ammonium |
| | | | | hydroxide |
| 9 | Q(1)[Ni(II)Cl₂(OH)(THF)₃] | NiCl₂ | I₂ | Benzyltrimethyl- |
| | I₂ | | | ammonium |
| | | | | hydroxide |
| 10 | Q(1)[Mn(II)Cl₂(OH)(THF)₃] | MnCl₂ | I₂ | Benzyltrimethyl- |
| | I₂ | | | ammonium |
| | | | | hydroxide |
| 11 | Q(1)[Pb(II)Cl₂(OH)(THF)₃] | PbCl₂ | I₂ | Benzyltrimethyl- |
| | I₂ | | | ammonium |
| | | | | hydroxide |
| 12 | Q(1)[Sn(II)Cl₂(OH)(THF)₃] | SnCl₂ | I₂ | Benzyltrimethyl- |
| | I₂ | | | ammonium |
| | | | | hydroxide |

(comp.) comparative example;
Q(1) = benzyltrimethylammonium with Q = N;
R₁ = benzyl,
R₂ = R₃ = R₄ = methyl

### Simulation Results for the Process for Producing Propylene Oxide by Oxidation of Propene in the Presence of the Epoxidation Catalyst System (1)

The activation energies for the catalytic reactions were calculated hereinafter by quantum-chemical simulations. For this purpose, a structure was drawn in each case according to the transition states A T1-B T2 (FIG. 1). The bonds drawn in bold were set at an atomic distance of in each case 1.90 Å (190 pm), and in the case of A T2 to 1.30 Å, and the structures thus obtained were converted to Cartesian coordinates. The atomic indices in the set of Cartesian coordinates of the bonds shown in bold in FIG. 3 were set as function space in the gradient-based Monte Carlo program, and the Monte Carlo procedure was executed until the corresponding transition states A T1-B T2 were obtained. Thereafter, the Cartesian coordinates of structures A T1-B T2 that were obtained in this way were manipulated in order to obtain the corresponding reactant catalyst complexes or the corresponding catalyst product complexes. For this purpose, one of the bonds drawn in bold in each case (FIG. 3) was i) extended and ii) shortened by ΔX=0.20 Å (20 pm). The structures i) and ii) thus obtained were expressed in the form of Cartesian coordinates and subjected to optimization of geometry by the DFT method described, and the resultant geometries were used for the calculation of the activation energies. The bonds selected in each case are compiled in table 3.

TABLE 3
Selection of bonds for manipulation
to obtain the equilibrium structures

| | Manipulated bond (marked in bold |
|---|---|
| Transition state | in FIG. 3, ΔX = 0.20 Å) |
| A T1 | carbon-oxygen |
| A T2 | alcoholic oxygen-hydrogen |
| A T3 | carbon-oxygen |
| B T1 | carbon-oxygen |
| B T2 | carbon-iodine |

FIG. 1: Three-stage reaction mechanism of the oxidative hydroxylation of propene with hydroxide under metal catalysis

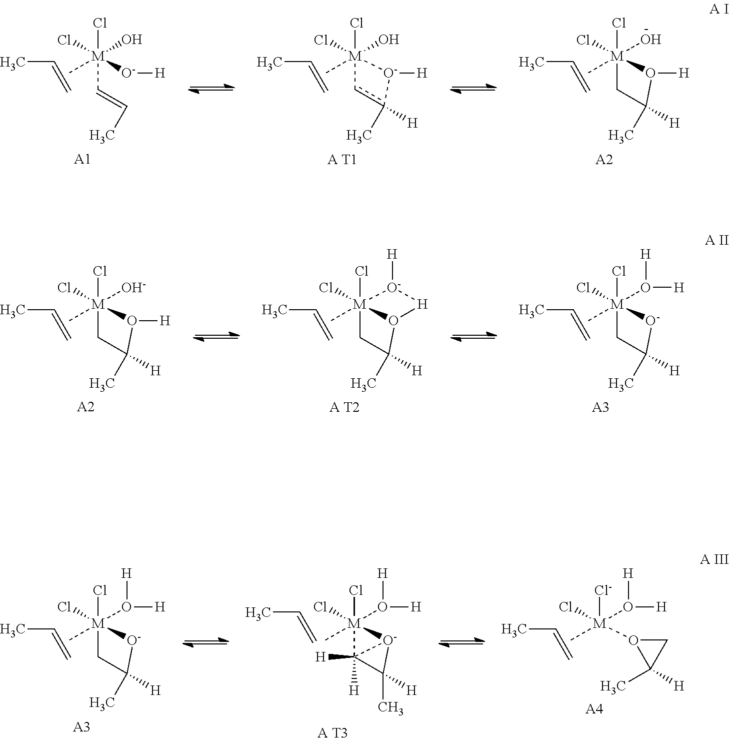

The three-stage mechanism described in FIG. 1 was calculated by quantum chemical simulation for various catalysts, and the activation energies from the respective reaction sequence were determined. The estimate of the activation energies thus obtained was used for assessment of various metal compounds as potential catalysts for oxidative hydroxylation. The results of the simulations are summarized in table 4.

TABLE 4
Comparison of the quantum-chemically simulated activation energies for the oxidative
conversion of propene with various epoxidation catalyst systems (1).

| | Epoxidation catalyst system | | | | |
|---|---|---|---|---|---|
| | (1) ^{a} | Eᵣₑₗ A T1 | Eᵣₑₗ A T2 | Eᵣₑₗ A T3 | E_{a, es} |
| Example | (Table 1, No.) | [kcal/mol] | [kcal/mol] | [kcal/mol] | [kcal/mol] |
| 13 | Q(1)[Ni(II)Cl(OH)₂(THF)₃] | 24.1 | — | 34.5 | too high |
| (comp.) | | | | | |
| 14 | [Cu(II)(OH)₂ (6) | 17.7 | — | 49.0 | too high |
| (comp.) | | | | | |
| 15 | Q(1)[Pt(II)Cl(OH)₂(THF)₂]⁻ | 45.9 | — | — | too high |
| (comp.) | | | | | |
| 16 | Q(1)[Ni(0)(OH) (THF)₂] | 35.2 | 15.5 | — | too high |
| (comp.) | | | | | |
| 17 | Q(1)[Co(II)Cl(OH)₂(THF)₂]⁻ | 27.2 | — | — | too high |
| (comp.) | | | | | |
| 18 | Q(1)[Mn(II)Cl(OH)₂(THF)₂] | 20.2 | — | 28.0 | too high |
| (comp.) | | | | | |
| 19 | [Mn(IV)Cl(OH)₃(THF)₂] (2) | 2.1 | 5.4 | 15.8 | 28.0 |
| 20 | [Nb(V)Cl(OH)₂(THF)₂]Cl₂ | 19.9 | — | 28.9 | 60.6 |
| (comp.) | | | | | |
| 21 | [Cr(VI)Cl₂(OH)₂(THF)₂]Cl₂ | Nc | 24.6 | 10.0 | 24.6 |
| | (3) | | | | |
| 22 | [Bi(V)C₁l₂(OH)₃ THF] (5) | 3.0 | nc | 13.6 | (13.6) |
| 23 | [Sn(IV)Cl₂(OH)₂(THF)₂] | 10.3 | 1.7 | 41.9 | too high |
| (comp.) | | | | | |
| 24 | [Pb(IV)Cl₂(OH)₂(THF)₂] (4) | 4.2 | 15.1 | 5.0 | 24.9 |
| 25 | [Sb(V)Cl₂(OH)₂(THF)₂]Cl | 1.6 | 18.0 | 19.9 | 31.7 |
| (comp.) | | | | | |
| 26 | [As(V)Cl₂(OH)₂(THF)₂]Cl | — | — | 38.3 | too high |
| (comp.) | | | | | |
| 27 | [Mo(V)Cl₂(OH)₂(THF)₂]Cl | 14.1 | 19.6 | 20.1 | 20.1 |
| | (1) | | | | |
| 28 | [V(V)Cl₂(OH)₂(THF)₂]Cl | — | — | deoxygenation | — |
| (comp.) | | | | | |
| 29 | [Ti(IV)Cl₂(OH)₂(THF)₂] (7) | — | — | deoxygenation | — |
| (comp.) | | | | | |

(comp.) comparative example;
Q(1) = benzyltrimethylammonium with Q = N;
R₁ = benzyl,
R₂ = R₃ = R₄ = methyl
^{a} Assuming that the THF ligand is at least partly displaced by propene from the complex epoxidation catalyst systems (1) before the oxidative conversion.

The results compiled in table 4 show that, in particular, the activation energies of the hydroxylation of propene and the reductive elimination of the propylene oxide formed depend significantly on the particular metal that functions as catalyst. This limits the possible selection of the metal compounds examined as catalysts for the oxidative hydroxylation to particular metals. Suitable metals are, in particular, Mo(V), Mn(IV), Cr(VI), Bi(V) and Pb(IV).

### Simulation Results for the Process for Producing Propylene Oxide by Oxidation of Propene in the Presence of the Epoxidation Catalyst System (2)

In a further execution B of the process, the epoxidation catalyst system (2) consisting of the metal salt (E), elemental iodine and the hydroxide (F) is used. This mixture (2) is laden with propene, firstly with addition of the hydroxide onto the double bond of propene. In a second step, iodine is then inserted into the metal-carbon bond, releasing an iodohydrin. This breaks down in the slightly basic medium to give propylene oxide and iodide, which is oxidized back to elemental iodine by supply of oxygen. The metal catalyst ensures controlled addition of hydroxide and iodide to give intermediate iodohydrin, without formation of predominantly unusable 1,2-diiodoalkanes. Compared to the known chlorine hydroxylation, iodine can be used here in catalytic or substoichiometric amounts. Thus, oxidation can be effected directly with oxygen.

For this purpose, quantum-chemical simulations were conducted in order to identify suitable metal catalysts.

TABLE 5
Comparison of the quantum-chemically simulated activation energies for
the iodine hydroxylation of propene with various metal catalysts

| | Epoxidation catalyst system (2)^{a)} | Eᵣₑₗ B T1 | Eᵣₑₗ B T2 | E_{a, es} |
|---|---|---|---|---|
| Example | (Table 1, No.) | [kcal/mol] | [kcal/mol] | [kcal/mol] |
| 30 (comp.) | Q(1)[Pt(II)Cl₂(OH)(THF)] I₂ (8) | — | 40.0 | too high |
| 31 | Q(1)[Ni(II)Cl₂(OH)(THF)] I₂ (9) | 0.0 | 18.1 | 25.5 |
| 32 | Q(1)[Mn(II)Cl₂(OH)(THF)] I₂ | 0.0 | 7.7 | 7.7 |
| | (10) | | | |
| 33 | Q(1)[Pb(II)Cl₂(OH)(THF)] I₂ (11) | 0.0 | 17.9 | 17.9 |
| 34 | Q(1)[Sn(II)Cl₂(OH)(THF)] I₂ (12) | 0.0 | 14.1 | 14.1 |

(comp.) comparative example;
Q(1) = benzyltrimethylammonium with Q = N;
R₁ = benzyl,
R₂ = R₃ = R₄ = methyl;
^{a)}Assuming that the THF ligand is at least partly displaced by propene and iodine from the complex epoxidation catalyst systems (1) before the oxidative conversion.

The simulation results compiled in table 5 show that the activation energy of

iodine insertion depends significantly on the metal catalysts used. Especially Mn(II), Sn(II) and Pb(II), but also Ni(II), are thereby identified as being suitable as catalysts for the reaction.

FIG. 2: Two-stage reaction mechanism for the metal-catalyzed iodine hydroxylation of propene

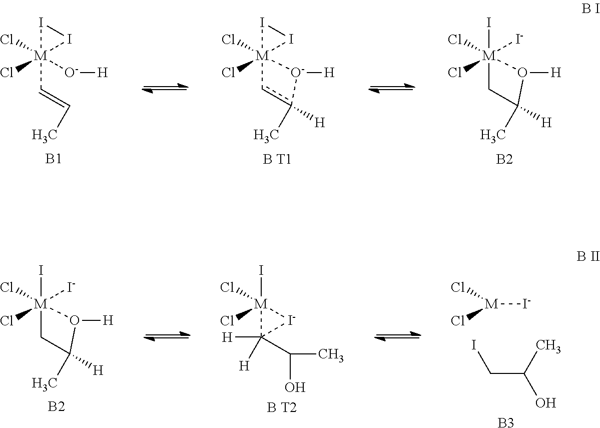

FIG. 3: Input geometries for the quantum-chemical calculations of the transition states of the catalyzed oxidative hydroxylations A and iodine hydroxylations B.

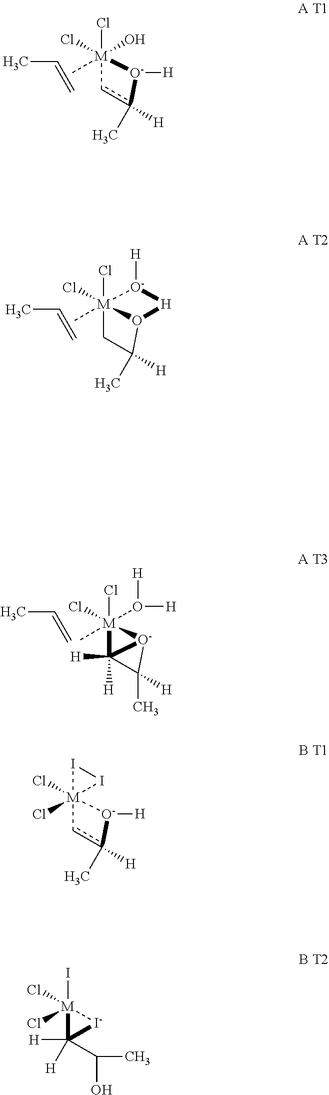

### General Experimental Procedure for the Process for Producing Propylene Oxide by Oxidation of Propene in the Presence of the Epoxidation Catalyst System (1) in a Batchwise Stirred Tank

A pressure-resistant 1 l reactor with stirrer system, pressure relief valve, pressure sensor, riser tube for removal of liquid, and sparging and degassing conduits was initially charged with 200 ml of tetrahydrofuran under inert conditions. Added to the solvent in each experiment were 0.020 mol of the metal chloride of the appropriate oxidation state (see table 1, 1-5) and 0.020 mol of CuCl2 under an opposing protective gas flow. While stirring vigorously, N-benzyltrimethylammonium hydroxide (trade name: Triton B) was in each case stirred in until a liquid sample taken, in contact with a water-moist pH test strip, gave a pH of 10-11. 0.400 mol of propene was then injected into the reaction vessel (about 11 bar). The internal reactor temperature was kept at 120° C. by closed-loop control, and oxygen was slowly injected until a molar amount of 0.200 mol of oxygen was attained. The addition of the oxygen was carried out such that the additional pressure rise was never more than 2 bar. The progress and endpoint of the reaction were ascertained from the pressure profile and/or by withdrawal of liquid and gas samples and analysis by GC analyzed. After the reaction had ended, the reactor was cooled down to 40° C., then expanded, and the propylene oxide reaction product was distilled off into a cooled receiver via the degassing conduit.

The yield of propylene oxide was 23.2 g for all five experiments.

### General Experimental Procedure for the Process for Producing Propylene Oxide by Oxidation of Propene in the Presence of the Epoxidation Catalyst System (2) in a Batchwise Stirred Tank

A pressure-resistant 11 reactor with stirrer system, pressure relief valve, pressure sensor, riser tube for removal of liquid, and sparging and degassing conduits was initially charged with a mixture of 180 ml of tetrahydrofuran and 20 ml of water under inert conditions. Added to the solvent mixture were 0.020 mol of the metal chloride of the appropriate oxidation state (see table 2, 9-12) and 0.040 mol (5.1 g) of elemental iodine under an opposing protective gas flow. While stirring vigorously, N-benzyltrimethylammonium hydroxide (trade name: Triton B) was stirred in until a liquid sample taken, in contact with a water-moist pH test strip, gave a pH of 10-11. 0.400 mol of propene was then injected into the reaction vessel (about 11 bar). The internal reactor temperature was kept at 120° C. by closed-loop control, and oxygen was slowly injected until a molar amount of 0.200 mol of oxygen was attained. The addition of the oxygen was carried out such that the additional pressure rise was never more than 2 bar. The progress and endpoint of the reaction were ascertained from the pressure profile and/or by withdrawal of liquid and gas samples and analysis by GC analyzed. After the reaction had ended, the reactor was cooled down to 40° C., then expanded, and the propylene oxide reaction product was distilled off into a cooled receiver via the degassing conduit.

The yield of propylene oxide was 23.2 g for all four experiments.

### General Experimental Procedure for the Process for Producing Propylene Oxide by Oxidation of Propene in the Presence of the Epoxidation Catalyst System (1) in a Continuous One-Bubble Column Reactor

A pressure-resistant bubble column (diameter 15 cm) with sparging frit, pressure relief valve, pressure sensor, upper and lower liquid withdrawal ports, upper addition point for solids, and inlets and outlets for gases was initially charged with 3500 ml of tetrahydrofuran under inert conditions. Added to the solvent in each experiment were 0.350 mol of the metal chloride of the appropriate oxidation state (see table 1, 1-5) and 0.350 mol of CuCl2 under an opposing protective gas flow. While sparging with N2 at 10 l/min, N-benzyltrimethylammonium hydroxide (trade name: Triton B) was added until a liquid sample taken, in contact with a water-moist pH test strip, gave a pH of 10-11. The bubble column was then brought to reaction temperature, 120° C., and a mixture of propene, oxygen and nitrogen (molar ratio of propene to oxygen 2:1) was introduced, with establishment of a total volume flow rate of less than 10 l/min by closed-loop control. The progress of the reaction and its steady-state point were ascertained by withdrawing liquid and gas samples at the upper withdrawal points and analyzing them by GC. The continuously generated product gas mixture was freed of propylene oxide by cascaded cooling, and the resulting gas stream was slightly compressed again and, in a blend with propene, oxygen and nitrogen, fed to the sparging frit of the bubble column.

Selectivities for propylene oxide were between 95% and 100% in all five experiments.

### General Experimental Procedure for the Process for Producing Propylene Oxide by Oxidation of Propene in the Presence of the Epoxidation Catalyst System (2) in a Continuous One-Bubble Column Reactor

A pressure-resistant bubble column (diameter 15 cm) with sparging frit, pressure relief valve, pressure sensor, upper and lower liquid withdrawal ports, upper addition point for solids, and inlets and outlets for gases was initially charged with 3150 ml of tetrahydrofuran and 350 ml of water under inert conditions. Added to the solvent mixture in each experiment were 0.350 mol of the metal chloride of the appropriate oxidation state (see table 2, 9-12) and 0.700 mol (89.3 g) of elemental iodine under an opposing protective gas flow. While sparging with N2 at 10 l/min, N-benzyltrimethylammonium hydroxide (trade name: Triton B) was added until a liquid sample taken, in contact with a water-moist pH test strip, gave a pH of 10-11. The bubble column was then brought to reaction temperature, 120° C., and a gas mixture of propene, oxygen and nitrogen (molar ratio of propene to oxygen 2:1) was introduced, with establishment of a total volume flow rate of less than 10 l/min by closed-loop control. The progress of the reaction and its steady-state point were ascertained by withdrawing liquid and gas samples at the upper withdrawal points and analyzing them by GC. The continuously generated product gas mixture was freed of propylene oxide by cascaded cooling, and the resulting gas stream was slightly compressed again and, in a blend with propene, oxygen and nitrogen, fed to the sparging frit of the bubble column.

Selectivities for propylene oxide were between 95% and 100% in all four experiments.

### General Experimental Procedure for the Process for Producing Propylene Oxide by Oxidation of Propene in the Presence of the Epoxidation Catalyst System (1) in a Continuous Two-Bubble Column Reactor

Two pressure-resistant bubble columns (diameter 15 cm), each equipped with sparging frit, pressure relief valve, pressure sensor, upper and lower liquid withdrawal/addition ports, upper addition point for solids, and inlets and outlets for gases are connected via a pump via the upper and lower liquid withdrawal points. One bubble column was initially charged with 3500 ml of tetrahydrofuran under inert conditions. Added to the solvent in each experiment were 0.350 mol of the metal chloride of the appropriate oxidation state (see table 1, 1-5) and 0.350 mol of CuCl2 under an opposing protective gas flow. While sparging with nitrogen at 10 l/min, N-benzyltrimethylammonium hydroxide (trade name: Triton B) was added until a liquid sample taken, in contact with a water-moist pH test strip, gave a pH of 10-11. The second bubble column was prepared analogously. The bubble columns were then brought to reaction temperature, 120° C. A gas mixture of nitrogen and propene was fed into the first bubble column, and a gas mixture of nitrogen and oxygen was fed into the second bubble column, with establishment of a total volume flow rate of less than 10 l/min by closed-loop control in both cases. The molar ratio of propene to oxygen was 2:1. At the same time, the liquid phase was withdrawn continuously from the first bubble column at the upper point and added to the second bubble column at the lower point, and withdrawn continuously from the second bubble column at the upper point and added to the first bubble column at the lower point. The volume flows were adjusted by closed-loop control such that there was no change in the fill levels of the two bubble columns over the duration of the experiment. The progress of the reaction and its steady-state point were ascertained by withdrawing liquid and gas samples at the upper withdrawal points of the two bubble columns and analyzing them by GC. The continuously generated product gas mixture from the first bubble column was freed of propylene oxide by cascaded cooling, and the resulting gas stream was slightly compressed again and, in a blend with nitrogen and propene, fed to the sparging frit of the bubble column. The gas stream from the second bubble column was freed of propene by cascaded cryogenic cooling, and disposed of via the air output. The propene recovered was reused by addition at the lower sparging point of the first bubble column.

Selectivities for propylene oxide were between 95% and 100% in all five experiments.

### General Experimental Procedure for the Process for Producing Propylene Oxide by Oxidation of Propene in the Presence of the Epoxidation Catalyst System (2) in a Continuous Two-Bubble Column Reactor

Two pressure-resistant bubble columns (diameter 15 cm), each equipped with sparging frit, pressure relief valve, pressure sensor, upper and lower liquid withdrawal/addition ports, upper addition point for solids, and inlets and outlets for gases are connected via a pump via the upper and lower liquid withdrawal points. One bubble column was initially charged with 3500 ml of tetrahydrofuran under inert conditions. Added to the solvent in each experiment were 0.350 mol of the metal chloride of the appropriate oxidation state (see table 2, 9-12) and 0.700 mol (89.3 g) of elemental iodine under an opposing protective gas flow. While sparging with nitrogen at 10 l/min, N-benzyltrimethylammonium hydroxide (trade name: Triton B) was added until a liquid sample taken, in contact with a water-moist pH test strip, gave a pH of 10-11. The second bubble column was prepared analogously. The bubble columns were then brought to reaction temperature, 120° C. A gas mixture of nitrogen and propene was fed into the first bubble column, and a gas mixture of nitrogen and oxygen was fed into the second bubble column, with establishment of a total volume flow rate of less than 10 l/min by closed-loop control in both cases. The molar ratio of propene to oxygen was 2:1. At the same time, the liquid phase was withdrawn continuously from the first bubble column at the upper point and added to the second bubble column at the lower point, and withdrawn continuously from the second bubble column at the upper point and added to the first bubble column at the lower point. The volume flows were adjusted by closed-loop control such that there was no change in the fill levels of the two bubble columns over the duration of the experiment. The progress of the reaction and its steady-state point were ascertained by withdrawing liquid and gas samples at the upper withdrawal points of the two bubble columns and analyzing them by GC. The continuously generated product gas mixture from the first bubble column was freed of propylene oxide by cascaded cooling, and the resulting gas stream was slightly compressed again and, in a blend with nitrogen and propene, fed to the sparging frit of the bubble column. The gas stream from the second bubble column was freed of propene by cascaded cryogenic cooling, and disposed of via the air output. The propene recovered was reused by addition at the lower sparging point of the first bubble column.

Selectivities for propylene oxide were between 95% and 100% in all four experiments.

### General Experimental Procedure for the Process for Producing Propylene Oxide by Oxidation of Propene in the Presence of the Supported Epoxidation Catalyst System (1) in a Continuous Gas Phase Reactor

A pressure-resistant 1 l reactor with stirrer system, pressure relief valve, pressure sensor, riser tube for removal of liquid, and sparging and degassing conduits was initially charged with a mixture of 200 ml of tetrahydrofuran under inert conditions. Added to the solvent mixture in each experiment were 0.350 mol of the metal salt (A) of the appropriate oxidation state (see table 1, example 1-5) and 0.350 mol of CuCl2 under an opposing protective gas flow. While stirring vigorously, N-benzyltrimethylammonium hydroxide as hydroxide (F) (trade name: Triton B) was stirred in until a liquid sample taken, in contact with a water-moist pH test strip, gave a pH of 10-11. Subsequently, the mixture obtained was applied to silica as support material. The solids were then introduced into an inertized pressure-resistant and pressure-safeguarded flow reactor. Nitrogen and oxygen were then passed through the flow reactor, which was brought to reaction temperature 180° C. Subsequently, a certain amount of propene was added to the gas flow until a molar ratio of propene to oxygen of 2:1 was attained, and contacted continuously with the solids. The flow conditions and contact time were chosen here such that partial conversions were attained. The continuously produced product gas mixture was in each case freed of propylene oxide by cascaded cooling, and the resulting gas stream was heated again and enriched with oxygen and propene, and the resulting mixture was introduced again into the solids. The gas is analyzed by taking of gas samples and analysis thereof by GC.

Selectivities for propylene oxide were between 95% and 100% in all five experiments.

1. An epoxidation catalyst system comprising:
a) a mixture or a reaction product of:
a-1) a metal salt of the metals chromium (Cr), manganese (Mn), molybdenum (Mo), lead (Pb) and/or bismuth (Bi); and
a-2) a hydroxide; and
b) a redox-active compound.

2. The epoxidation catalyst system as claimed in claim 1, wherein the metal salt is one or more compounds and comprises MoCl₅, MoOCl₃, MnCl₂, K₂MnCl₆, CrOCl₂, PbCl₄, BiCl₃, or a combination thereof.

3. The epoxidation catalyst system as claimed in claim 1, wherein the hydroxide is one or more compound(s) and comprises (trifluoromethyl)phenyltrimethylammonium hydroxide, tetra-n-butylphosphonium hydroxide, choline hydroxide, benzyltrimethylammonium hydroxide, tetraethylammonium hydroxide, tetra-n-propylammonium hydroxide, tetrabutylammonium hydroxide, tetramethylammonium hydroxide, or a combination thereof.

4. The epoxidation catalyst system (1) as claimed in claim 1, wherein the redox-active compound is one or more compound(s) and comprises CuCl₂, Cu(BF₄)₂, CuCl, VCl₃, VOCl₃, NH₄VO₃, 1,4-benzoquinone, 1,4-naphthoquinone, Se₂O₅, TeO₂, TeO₂, Sb₂O₃, Sb₂O₅, CeCl₃, Co(salen), Co(OAc)₂, SnSO₄, Fe(acac)₃, Mo(acac)₃, K₂Cr₂O₇, Mn(OAc)₃, Ni(CF₃CO₂H)₂, BiCl₃, or a combination thereof.

5. The oxidation catalyst system as claimed in claim 1, wherein the epoxidation catalyst system comprises the reaction product, and the reaction product has a structure of formula (I), (II) and/or (III):

| | | |
|---|---|---|
| Q R₁R₂R₃R₄)⁺ₙ₊ₒ₋ₘ[M(A)^{m+}(Hal)ₙ(OH)ₒ(S)ₚ]^{m-n-o} | (I) | if m < n + o |
| [M(A)^{m+}(Hal)ₙ(OH)ₒ(S)ₚ] | (II) | if m = n + o |
| [M(A)^{m+}(Hal)ₙ(OH)ₒ(S)ₚ]^{(m-n-o)} [X]⁻ₙ₊ₒ₋ₘ | (III) | if m > n + o |

wherein
Q=nitrogen or phosphorus;
R₁, R₂, R₃, and R₄ are each independently;
(i) a linear or branched alkyl group containing 1 to 22 carbon atoms, optionally substituted by heteroatoms and/or heteroatom-containing substituents;
(ii) a cycloaliphatic group containing 3 to 22 carbon atoms with 1 to 3 bridging carbon atoms, optionally substituted by heteroatoms and/or heteroatom-containing substituents; and/or
(iii) an aryl group containing 6 to 18 carbon atoms, optionally substituted by 1 to 10 carbon atoms and/or optionally substituted by heteroatoms and/or heteroatom-containing substituents;
M(A)^{m+}=Mo⁵⁺, Mn⁴⁺, Cr⁴⁺, Pb⁴⁺, or Bi⁵⁺;
Hal=Cl⁻, Br⁻ or I⁻;
S=H₂O, THF (tetrahydrofuran), dioxane, bis(2-methoxyethyl) ether (diglyme), methoxyethanol, a polyethylene glycol, pyridine, lutidine, 2,2′-bipyridine, acetonitrile, dimethyl sulfoxide, sulfolane, or thiophene;
n+o+p=6;
n≥1;
o≥1;
X=OTf⁻, BF4⁻, or Hal⁻.

6. An epoxidation catalyst system comprising:
a) a mixture or a reaction product of:
c-1) a metal salt,
c-2) iodine (I₂), and
c-3) a hydroxide; and
b) optionally a redox-active compound.

7. The epoxidation catalyst system as claimed in claim 6, wherein the metal salt is one or more compounds and comprises NiCl₂, MnCl₂, PbCl₂, SnCl₂, CrCl₃, VCl₃, MoCl₄, FeCl₂, RuCl₃, or a combination thereof.

8. The epoxidation catalyst system as claimed in claim 6, wherein the hydroxide is one or more compound(s) and comprises 3-(trifluoromethyl)phenyltrimethylammonium hydroxide, tetra-n-butylphosphonium hydroxide, choline hydroxide, benzyltrimethylammonium hydroxide, tetraethylammonium hydroxide, tetra-n-propylammonium hydroxide, tetrabutylammonium hydroxide, tetramethylammonium hydroxide, or a combination thereof.

9. The oxidation catalyst system as claimed in claim 6, wherein the epoxidation catalyst system comprises the reaction product, and the reaction product has a structure of formula (IV), (V) and/or (VI):

| | | |
|---|---|---|
| (Q R₁R₂R₃R₄)⁺_{Im-o-pI} [M(E)^{m+}(Hal)ₙ(OH)ₒ(S)ₚ]^{m-n-o} · q · I₂ | (IV) | if m < n + o, |
| [M(E)^{m+}(Hal)ₙ(OH)ₒ(S)ₚ] · q · I₂ | (V) | if m = n + o |
| [M(E)^{m+}(Hal)ₙ(OH)ₒ(S)ₚ]^{m-n-o} · q · I₂ [X]⁻ₙ₊ₒ₋ₘ | (VI) | if m > n + o |

wherein
Q=nitrogen or phosphorus,
R₁, R₂, R₃, and R₄ are each independently:
(i) a linear or branched alkyl group containing 1 to 22 carbon atoms, optionally substituted by heteroatoms and/or heteroatom-containing substituents;
(ii) a cycloaliphatic group containing 3 to 22 carbon atoms with 1 to 3 bridging carbon atoms, optionally substituted by heteroatoms and/or heteroatom-containing substituents; and/or
(iii) an aryl group containing 6 to 18 carbon atoms, optionally substituted by 1 to 10 carbon atoms and/or optionally substituted by heteroatoms and/or heteroatom-containing substituents;
M(E)^{m+}=Ni²⁺, Mn²⁺, Pb²⁺, Sn²⁺, Cr³⁺, V³⁺, Mo⁴⁺, Fe²⁺ or Ru³⁺;
Hal=Cl⁻, Br⁻ or I⁻;
n+o+p=6;
S=H₂O, THF (tetrahydrofuran), dioxane, bis(2-methoxyethyl) ether (diglyme), methoxyethanol, a polyethylene glycol, pyridine, lutidine, 2,2′-bipyridine, acetonitrile, dimethyl sulfoxide, sulfolane, or thiophene;
n≥1;
o≥1;
X=OTf⁻, BF₄⁻, or Hal⁻; and
q≥1.

10. A process for producing an epoxide, comprising oxidatively converting an alkene in a reactor in the presence of the epoxidation catalyst system as claimed in claim 1.

11. The process as claimed in claim 10, wherein the oxidative conversion in the reactor is effected in the presence of oxygen or an oxygen-containing gas mixture.

12. The process as claimed in claim 10, wherein the alkene is one or more compound(s) and comprises ethene, propene, butene, 1-octene, butadiene, butane-1,4-diol diallyl ether, allyl chloride, allyl alcohol, styrene, cyclopentene, cyclohexene, phenyl allyl ether, diallyl ether, n-butyl allyl ether, tert-butyl allyl ether, bisphenol A diallyl ether, resorcinol diallyl ether, triphenylolmethane triallyl ether, cyclohexane-1,2-dicarboxylic acid bis(allyl ester), isocyanuric acid tris(prop-2,3-ene) ester, or a mixture thereof.

13. The process as claimed in claim 10, wherein the preparation is effected in the presence of a solvent.

14. The process as claimed in claim 10, wherein the preparation is effected in the absence of a solvent.

15. The process as claimed in claim 10, wherein the alkene and the oxygen-containing gas mixture are metered into the reactor continuously or stepwise.

16. A process for producing an epoxide, comprising oxidatively converting an alkene in a reactor in the presence of the epoxidation catalyst system as claimed in claim 6.

17. The process as claimed in claim 16, wherein the oxidative conversion in the reactor is effected in the presence of oxygen or an oxygen-containing gas mixture.

18. The process as claimed in claim 16, wherein the alkene is one or more compound(s) and comprises ethene, propene, butene, 1-octene, butadiene, butane-1,4-diol diallyl ether, allyl chloride, allyl alcohol, styrene, cyclopentene, cyclohexene, phenyl allyl ether, diallyl ether, n-butyl allyl ether, tert-butyl allyl ether, bisphenol A diallyl ether, resorcinol diallyl ether, triphenylolmethane triallyl ether, cyclohexane-1,2-dicarboxylic acid bis(allyl ester), isocyanuric acid tris(prop-2,3-ene) ester, or a mixture thereof.

19. The process as claimed in claim 16, wherein the preparation is effected in the presence of a solvent.

20. The process as claimed in claim 16, wherein the preparation is effected in the absence of a solvent.
